(19) United States
(12) Patent Application Publication
(10) Pub. No.: US 2026/0283572 A1
(43) Pub. Date: Sep. 24, 2026

(54) MICROSPHERE DELIVERY APPARATUS PROVIDING REAL-TIME MEASURING OF DELIVERED RADIOACTIVITY

(71) Applicant: Beth Israel Deaconess Medical Center, Inc., Boston, MA (US)

(72) Inventors: Ammar Sarwar, Jamaica Plain, MA (US); Joseph Ring, Green Harbor, MA (US)

(21) Appl. No.: 19/474,603

(22) PCT Filed: Apr. 11, 2024
(86) PCT No.: PCT/US2024/024144

§ 371 Date: Oct. 10, 2025

Related U.S. Application Data
(60) Provisional application No. 18/133,962, filed on Apr. 12, 2023.

(51) Int. Cl. A61B 6/40; A61B 6/00; A61B 6/46; A61K 51/12; A61M 5/00; A61M 5/168; A61M 5/172

(52) U.S. Cl. CPC A61B 6/4057; A61B 6/461; A61B 6/563; A61K 51/1255; A61M 5/007; A61M 5/16886; A61M 5/1723

(57) ABSTRACT

A microsphere delivery apparatus providing real-time measuring of delivered radioactivity during procedures utilizing radioactive emitting microspheres wherein an administration housing includes a holder to receive a vial containing microspheres for delivery from a housing, a detector holder to receive a radiation detector and a delivery tube assembly for transporting fluid containing the microspheres.

### BACKGROUND

This is a continuation-in-part application of U.S. patent application Ser. No. 18/133,962 filed on Apr. 12, 2023, the entire contents of which are incorporated herein by reference.

Conventionally, radioactive microspheres are delivered in a delivery vial by a manufacturer to a nuclear medicine (NM) department. Typically, the use of stationary dose calibrators in the NM department is the initial point of radioactivity measurement. The NM department then draws microspheres with a prescribed amount of radioactivity from the vial depending on the needs of the patient. This drawn dose is then delivered to the angiography suite for injection into the patient, for example during Selective Internal Radiation Therapy (SIRT) and Transarterial Radioembolization (TARE) procedures. However, many challenges arise at the time of dose draw that makes ⁹⁰Y radiation microsphere administration (and administration of other types of impregnated microspheres) complex. For example, the American Association of Physicists in Medicine (AAPM) recommends a minimum number of parallel vial ruptures to prevent leakage but those can be subjective and prone to operator error. This is corroborated by findings from medical reports by Nuclear Regulatory Commission (NRC) in which intersection of multiple puncture paths into each vial resulted in increased risk of vial leakage and reduction in amount of prescribed radioactivity being delivered. Another challenge is that operator experience and skill for the nuclear medicine technologist can vary between institutions with such requirements leading to potential inconsistent practices, further adding to the risk of radioactivity spillage and uncertainty in the amount of administered radioactivity. Moreover, the practice of interventional radiology is moving towards the use of outpatient office-based laboratories, which do not have an attached NM department, thus reducing access to patients at these facilities. Further, the process of repeating dose draws to ensure withdrawn radioactivity matches a written directive is a time-consuming and laborious process. This process becomes more tedious for smaller activities, when multiple TARE procedures are scheduled on the same day, or a single patient requires two different activities.

Owing to an increasing number of SIRT microsphere treatments being performed in the last decade, the number of adverse Medical Events (MEs) have also significantly increased. A NRC ME report (2008-2017) showed that 85% of ME's were due to prescribed dose deviation of which, about 75% were due to less than prescribed doses being delivered. While some cases were due to microsphere settling and device clogging, most of the time, the medical teams found these procedural discrepancies incidentally. Often, medical teams, including a manufacturer's representative, did not identify any abnormalities during the procedure, and it was only post-procedurally that a radiation safety check confirmed that the actual radioactivity delivered was less than what was prescribed. This was also highlighted in a 2022 meeting of NRC's Advisory Committee on Medical Uses of Isotopes (ACMUI) where most causes of microsphere under dosing were found at the post-procedure survey and inspection. Thus, knowledge of the amount of radioactivity delivered in the catheterization laboratory to each patient is crucial to ensure desired radioactivity and threshold target doses are achieved. However, in the standard assessment methodology, the administered radioactivity is estimated after the procedure in nuclear medicine departments by comparing the radioactivity in the prepared vial to the residual radioactivity in the vial after the therapy session and radioactive waste after administration.

### BRIEF SUMMARY

Embodiments of the present invention allow an interventional radiologist operator to monitor radioactivity being delivered to a patient from a microsphere vial, or other container configured to house radioactive particles, in real-time during a medical procedure. Radioactive isotopes can be delivered into patients with a particulate carrier that serves to deliver radioactive isotopes into a patient with a delivery system. More particularly, the radioactive particles can be in the form of microspheres that are manufactured to a specific size and capacity for retaining radioactive isotopes as described herein. The microsphere delivery apparatus can include an administration housing enclosing both a delivery apparatus and a radioactivity detector system. The delivery apparatus enables radioactive microspheres, for example, to be delivered in a targeted manner to anatomic features of a patient during TARE and other medical procedures. The administration or delivery housing is configured to contain any leakage of radioactive material along the delivery path from the radioactive particle container to the patient. The detector system is configured to perform real-time monitoring of the amount of radioactivity being delivered to the patient from the microsphere vial during the procedure. The detector system includes both a radiation probe and signal processing circuitry such as a scaler connected to an electronic display configured to display outputs from the detector in units of radioactivity. Since the microsphere delivery apparatus enables the interventional radiologist operator to be aware of the precise amount of radioactivity associated with the microspheres during delivery where the microspheres are contained in a vial that is initially received in the angiography suite, the interventional radiologist operator can continually monitor the amount of radioactivity being administered to the patient and adjust accordingly to meet prescribed dose levels.

Embodiments can be used for the treatment of liver cancer, for example, in which radioisotopes such as yttrium (⁹⁰Y), holmium (¹⁶⁶Ho) or other suitable isotopes (see Bouvry et al; Transarterial Radioembolization (TARE) Agents beyond ⁹⁰Y-Microspheres, Biomed Research International; Vol. 2018, the entire contents thereof being incorporated herein by reference) can be attached to or encapsulated within microspheres at a known dosage and half life for delivery to a patient. Microspheres are advantageous in that the size and characteristics can be selected to enhance delivery of the desired dose of radiation to a specific tissue location. Microspheres such as those fabricated with poly lactic-co-glycolic acid (PLGA) can be biodegradeable, and thus can be used to deliver to a specific site and then degrade after use. The microspheres can serve to occlude vessels leading to a tumor and thereby reduce the growth of the tumor. Lower molecular weight PLGA can have a faster degradation rate and thus enable selection of the period for occlusion after delivery, for example. A housing containing the delivery vial with a prescribed dose, a radiation detector and at least a portion of a disposable delivery tube assembly is used to manage delivery of the therapy. One or more manual or robotically operated syringes can be used to actuate flow of fluid within the delivery system to deliver fluid that transports the radioactive isotopes through a catheter and into the patient. The delivery housing can include a first receiving region or container (e.g. vial) holder in which a vial containing the microspheres is positioned for use. A second receiving region or detector holder is configured to receive the radiation detector which can be placed inside the container and removed after use. Alternatively, the housing can have an opening in an outer wall so that the radiation detector can be inserted through the opening and into the detector holder. Thus, the housing provides a fixed relationship between the vial, the detector and the delivery tube assembly to provide a simple and repeatable procedure for the user. The housing also serves to capture any spilled fluid that may contain radioactive microspheres and thereby improve safety during use.

The radiation detector is electrically connected to an interface having a memory that records the measured radioactivity level during use and can also be connected to an electronic display where the radioactivity level and other procedural information can be displayed to the user. The interface can also include a network interface to transport data for storage in a local or an external memory device and delivery to the electronic medical record of the patient receiving the treatment. The system and/or the interface can thus include one or more data processors and/or controllers to manage data processing and control functions. A software program can be configured to control operations of the system including the display and network interfaces and to log data retrieved from the detector to a memory whereby the results for each patient can be stored in the patient's electronic medical record. The system can generate a data record for the delivery procedure including one or more frames of data that can include a plurality of fields including patient identification data, time stamp data for each frame and activity level data delivered during a selected time interval. A user can enter patient information into the system including information for treatment planning, scheduling, administration and to generate medical records for the treatment and any follow-up and monitoring of the patient as well as any further treatment subsequent to a treatment session. The software for this system can include modules including a treatment module and a monitoring module to verify the delivered radioactivity.

Embodiments of the housing can include an external connector so that the interface components can be plugged into the external connector and thereby receive signals from the detector assembly. Alternatively, the interface and the display can be integrated into the housing, or attached to external surfaces on the housing. The system can interface with a mobile phone or tablet device that allows a user to monitor the procedure during treatment and/or observe the results. With such a delivery housing, the interventional radiologist operator can monitor radioactivity and administer the precise amount of radioactive microspheres from the microsphere vial according to the needs of the patient. Further, embodiments inform interventional radiologists of the completion of radioactivity delivery compared to the current use of visual estimation of microsphere delivery level. Thus, operators can confirm the precise amount of radioactivity delivered to the patient before terminating the procedure when the required radioactivity has been delivered from the vial. In further embodiments, for patients in which two or more delivery sites are required, the system can be programmed to track the sequence and prompt the user regarding each step of the process. The display can be programmed to show the total radioactivity cumulatively measured over time or graphically depict the increasing amount being delivered until the prescribed total is reached at a displayed level. An alarm can indicate to the user when the prescribed level is reached. The amount remaining in the vial can also be indicated and recorded.

Embodiments of the presently described system thus provide apparatus and methods for monitoring transfer of radiation emitting particles using fluids as a transfer medium in a laboratory or clinic. The housing can provide a containment system for the safe management of radioactive materials that records the amount of microspheres transferred from the source of the microspheres and thereby accurately tracks the disposition of this material from the container during a sequence of transfer periods. The system can provide a programmable automated system to control the transfer process and electronically record and communicate a data record of the process to comply with regulatory requirements.

### BRIEF DESCRIPTION OF THE DRAWINGS

The accompanying drawings, which are incorporated in and constitute a part of this specification, illustrate one or more embodiments of the invention and, together with the description, help to explain the invention. In the drawings:

FIG. 1A depicts a microsphere delivery apparatus providing real-time measuring of delivered radioactivity in an exemplary embodiment;

FIG. 1B depicts a microsphere delivery apparatus providing real-time measuring of delivered radioactivity wherein the display is integrated into, or mounted on, the housing in another exemplary embodiment;

FIG. 1C shows an exemplary circuit diagram for an embodiment of a detector system.

FIG. 2 is a flowchart of an exemplary sequence of steps used to provide real-time measuring of delivered radioactivity in an exemplary embodiment;

FIG. 3 depicts fused PET/CT images obtained approximately one hour after intravenous administration of microspheres demonstrating two areas of radioactivity in an exemplary trial;

FIG. 4 depicts GMC radioactivity readings delivered with each push from of vials with varying starting radioactivity levels in an exemplary trial; and

FIG. 5 depicts the number of pushes and operator variability in delivering>90% 1 GBq of vial radioactivity in an exemplary trial.

### DETAILED DESCRIPTION

Embodiments of the present invention provide apparatus and methods for real-time monitoring of delivered radioactivity during TARE and similar procedures utilizing particulate carriers such as microspheres impregnated with radioactive emitting isotopes delivered to patients.

In the United States, the use of yttrium-90 (⁹⁰Y) for example, a pure β-emitting radioactive isotope used for SIRT/TARE procedures, is regulated under licensing guidance (Code of Federal Regulations (CFR) titled 10 CFR 35.1000) by the Nuclear Regulatory Commission (NRC). ⁹⁰Y microspheres are handled by experienced Authorized Users (AUs). The process of ⁹⁰Y microsphere administration requires a series of steps to be performed as detailed by the American Association of Physicists in Medicine (AAPM) as well as in manufacturer guidelines. Radioactivity measurement at end-user sites typically use dose calibrators that are calibrated to manufacturer settings to ensure that the prescribed amount of radioactivity in a written directive is transferred in the delivery container or vial.

These SIRT/TARE treatments do not always occur exactly as planned and challenges can arise at various stages (e.g., drawing appropriate doses or delivering microspheres in the presence of vascular stasis, deviations from prescribed doses, etc.). ME reports by the Advisory Committee on the Medical Uses of Isotopes (ACMUI) showed that between 2016-2021, 50-64% of annual MEs occurred due to ⁹⁰Y microsphere handling. In 2021, 92% of MEEs were due to over or under dosing, of which 84% had a root cause discovered on a post-treatment survey. This suggests that AUs are unable to confirm the accuracy of the radioactivity received in the delivery vial nor the changes in radioactivity during administration while in the angiography suite. Note that the isotopes decay over time, and consequently any delays experienced in delivery to a patient can result in deviation from the prescribed treatment.

To address these issues, embodiments of the present invention provide a microsphere delivery apparatus with real-time radioactivity monitoring and display capabilities. The use of microspheres can impact the aspects of treatment as the microspheres currently used to deliver yttrium can have a size in the range of 15-60 microns, for example, do not degrade over time and thus form a permanent implant at the delivery site. These microspheres are delivered to the liver to treat cancers of the liver, for example, with a catheter placed into the hepatic arteries and can serve to clog the smaller tumor capillaries that can be 8-10 microns in diameter. This can increase the concentration of microspheres delivered into and residing at the tumor site. Examples of such microspheres can include those available from Sirtex Medical Limited (SIR-Spheres) which are a resin comprising a sulfonated divinyl benzene-styrene copolymer and can be about 32 microns in diameter, for example. A dose can be specifically fabricated for each patient and can comprise millions of microspheres depending on the specific radioactivity needed to treat the tumor size and location. Particle sizes can be adapted for the specific delivery application required for diagnosis and/or treatment. Embodiments can also employ glass microspheres such as those available from Boston Scientific Corp. in Marlborough, MA (Therasphere™) that have a range of sizes of 20-30 microns in which ⁹⁰Y is embedded in a glass matrix which enables emission over a longer period of time than resin microspheres with the radioisotope adhering to the outer surface. Therasphere microspheres can provide a longer shelf life of up to 12 days, for example.

Holmium microspheres are available from Terumo Interventional Systems (QuiremSpheres™ available from Quirem Medical BV) for a delivery system, for example, that is being used for the treatment of lung cancer. The use of a 60 Gy dose of ¹⁶⁶Ho has been shown to be safe and effective for the treatment of liver cancer, for example. See Reinders, et al, Holmium-166 Microsphere Radioembolization of Hepatic Malignancies; Seminars in Nuclear Medicine; Vol. 16, Issue 3; May 2019, pgs 237-243; the entire contents of which is incorporated herein by reference. Magnetic resonance imaging (MRI) and single-photon emission computed tomography (SPECT) can be used to image and quantify perfusion and function of the holmium isotope after delivery. As these holmium microspheres also emit low energy gamma photons, this enables the visualization of distribution within the body after delivery to provide quantitative imaging. Holmium is also paramagnetic to provide for the use of MRI to monitor treatment progression and efficacy. The microspheres can be fabricated with poly-L-lactic acid and can have a size in the range of 15-60 microns. This can provide a particle density close to the density of blood for delivery through arteries to portions of the liver, and as these holmium microspheres biodegrade over time, this better enables the effectiveness of any subsequent radioembolization procedures.

Typically, an AU monitors delivery by visual estimation of ⁹⁰Y microspheres in the vial, the degree of back pressure against the syringes, angiographic flow rates, and the amount of remaining intravenous solution (such as 5% dextrose in water (D5W)) volume. Confirmation is drawn post-procedurally by a radiation survey by Nuclear Medicine and Radiation Safety at which time corrections to deviations in therapy may require repeat catheterization. Therefore, to aid in real-time evaluation of received and delivered radioactivity, this study used a portable GMC for real time radioactivity monitoring during radioembolization and compared these measurements to the standard method using dose calibrators.

In contrast to conventional approaches, embodiments of the present invention enable AUs to monitor delivered radioactivity in real-time. In one embodiment, a radiation delivery measurement apparatus includes an administration housing that includes a delivery apparatus and a detector. The delivery apparatus is configured to deliver radiation microspheres via tubing that can define a delivery path from a microsphere vial to a patient during SIRT. The detector is configured to perform real-time monitoring of administered radioactivity delivered to a patient during SIRT and includes a radiation probe for detecting radioactivity and a scaler configured to record and display an output of the radiation probe in units of radioactivity.

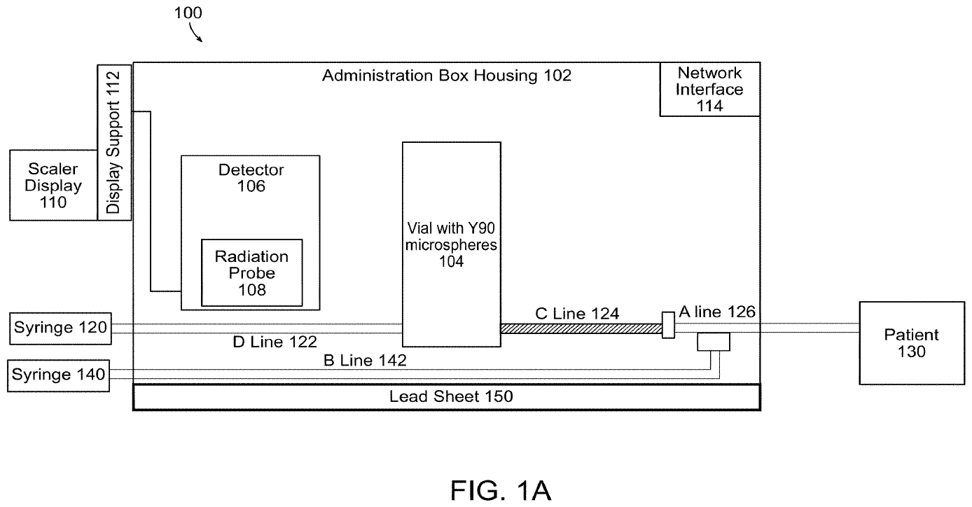

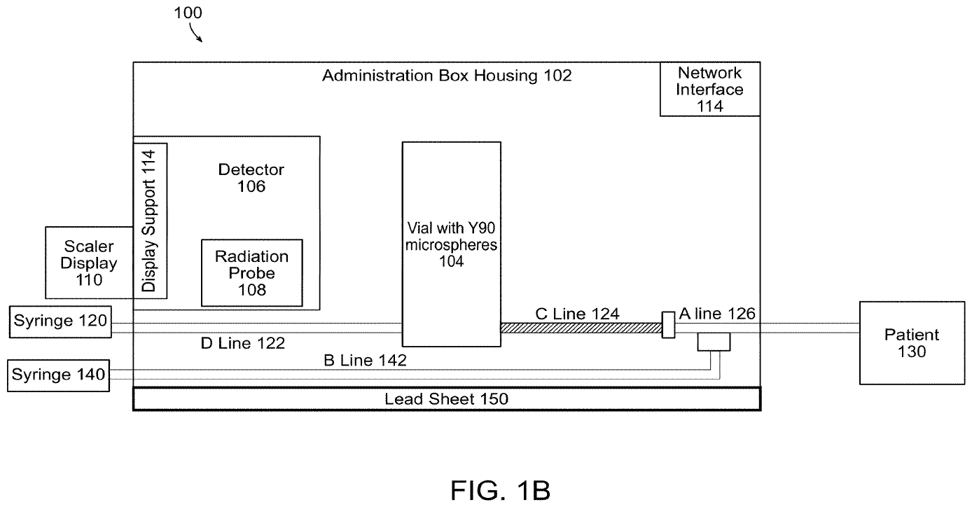

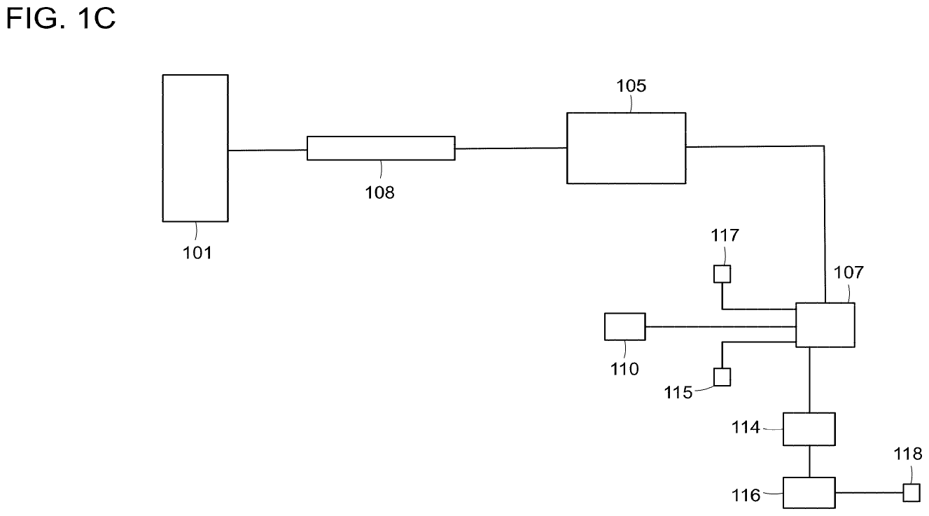

FIG. 1A-1C depict embodiments for a microsphere delivery apparatus providing real-time measuring of delivered radioactivity. An exemplary microsphere delivery apparatus 100 includes an administration or delivery housing 102. Administration housing 102 encloses a delivery apparatus and radioactivity detector. For example, the housing 102 may include a delivery apparatus including vial 104 containing microspheres impregnated with radioactive isotopes. The microspheres for example may be glass or resin or other type of radiation microspheres. In one embodiment, the radioactive isotope is ⁹⁰Y. In another embodiment the radioactive isotopes are holmium 166 (¹⁶⁶Ho). Connected to vial 104 may be an input tubing or first channel (D Line 122) and output tubing (C Line 124). A syringe 120 connected to D Line 122 may be used by an interventional radiologist or other operator to inject a delivery fluid (D5W set forth below) fluid into vial 104. The pressure from the injection causes the D5W fluid and a portion of the microspheres 104 to exit vial 104 into C Line or second channel 124. C Line 124 may be connected by microcatheter 126 (A Line 126) the distal end of which is inserted into patient 130 to deliver the radioactive microspheres to a targeted location such as an hepatic artery conveying the fluid with the entrained radioactive particles to a region in the patient to be treated, such as a tumor. Thus, the delivery path includes the tubing or second channel 124 from the vial 104 and the tubing or channel 126 that can comprise or include a catheter for delivery to the patient. Thus, a proximal end of catheter 126 can be coupled at a valve to second channel 124. Also included in the delivery apparatus is a mechanism for delivering contrast agent to the patient to assist in medical imaging. For example, administration housing box 102 may partially enclose input tubing (B Line 142) used to deliver contrast agent with syringe 140 to patient 130. In one embodiment, B Line or third channel 142 may connect via a valve that connects to the microcatheter, namely, at A Line 126. In certain embodiments, electronically operated pumps (at 120, 140) can be used instead of one or both syringes. Pressure and/or flow rate sensors 117 can be used to monitor fluid pressure and flow rates which can also be used to automatically control the flow and thus the delivery rate of radioactivity into the patient. A system processor or controller can be programmed to provide adjustable and/or automated flow control based on sensor data thereby providing feedback control of delivery rates over time.

Administration housing 102 also includes detector 106 for detecting radioactivity of the microspheres administered from vial 104. Detector 106 and radiation probe 108 are placed in sufficient proximity to the vial or container to accurately measure the activity over time. As the microspheres are carried out of container 104, the detector will record the decrease in activity. Optionally, a further radiation detector can be positioned relative to channel 124 along the delivery path or elsewhere within or external to housing 102 that can record data regarding activity in the delivery channel that does not reach the patient or to monitor safety of users and the patient. In one embodiment, detector 106 may be a Geiger-Mueller counter or other radiation measurement device. Geiger counters are commercially available from numerous manufacturers such as Thermo Scientific, for example, which typically include a gas filled tube, signal processing circuitry and a high voltage source. These detectors can be battery powered and can include a display in the detector housing to display radioactivity as a function of time. Detector 106 includes a radiation probe and a scaler and is configured to measure, record and display radioactivity of the microspheres administered from vial 104 in units of radioactivity. For example, the measured radioactivity may be presented via scaler display 110. In one embodiment, display 110 may be connected to administration box housing 102 via display support 120. Display support 120 may be, for example, a suction cup or extendable mechanical arm.

In some embodiments some or all of the walls of administration housing 102 may be partially or fully transparent to enable a user to observe the transition of fluids and any leakage of fluid that may occur. The housing and the delivery tube assembly can comprise disposable components in certain embodiments. A holder within the administration housing having an opening of less than 2.5 inches×2.5 inches, or an area less than 50 square centimeters can be used to install the detector into a holder within the housing that is within a volume of less than about 500 cubic centimeters.

In an embodiment, administration housing may include, or be connected to, network interface 114 that is communicatively coupled to detector 106 to enable captured data to be relayed in a wired or wireless manner to external computing 116 and data storage 118 devices for further processing, storage and/or display. The communication of the data to an offsite location allows the entire procedure's data to be studied and/or transferred into a medical records system.

In one embodiment, administration housing 102 may further include lead sheet 150 affixed to one or more walls to limit backscatter interference. In an embodiment, the lead sheet may have a thickness of approximately ⅛ of an inch.

FIG. 1B depicts a microsphere delivery apparatus providing real-time measuring of delivered radioactivity in another exemplary embodiment. As depicted in FIG. 1B instead of being affixed to administration box housing 102 by display support 112 as in FIG. 1A, scalar display 110 may insert into a display port or interface that is connected to detector 106.

FIG. 1C illustrates an exemplary circuit diagram wherein high voltage source 101 is connected to Geiger Mueller tube 108 wherein the anode and cathode are connected to pulse counter 105 which generates output signals processed by microcontroller 107. Local memory 115 can be used to store and log data. An optional sensor 117 may be provided to monitor flow rates and/or fluid pressure or can comprise a second hand held radioactivity detector for use with the system. Display 110 can display activity levels in real time to the user administrating or monitoring the infusion of the microspheres. The microcontroller can also control transfer of data to a network using network interface 114. Commercial devices incorporating these features are available from Analog Devices, Inc., Norwood, MA. See Circuit Note CN-0536, Analog Devices Inc, for example, the entire contents of which is incorporated by reference. Other detectors as described herein can generate an output connected to an amplifier wherein the amplified output signal is converted to a digital signal by an analog to digital converter (ADC). A processor or microcontroller then stores the data to a memory, outputs the result through the network interface or displays the data to a user. A field programmable gate array (FPGA) can be connected to the processor at 107 can also be used to further analyze, process and configure the digital data for storage at memory 115 or for export using network interface 114 to an external computer, electronic storage or the hospital or clinic medical record system.

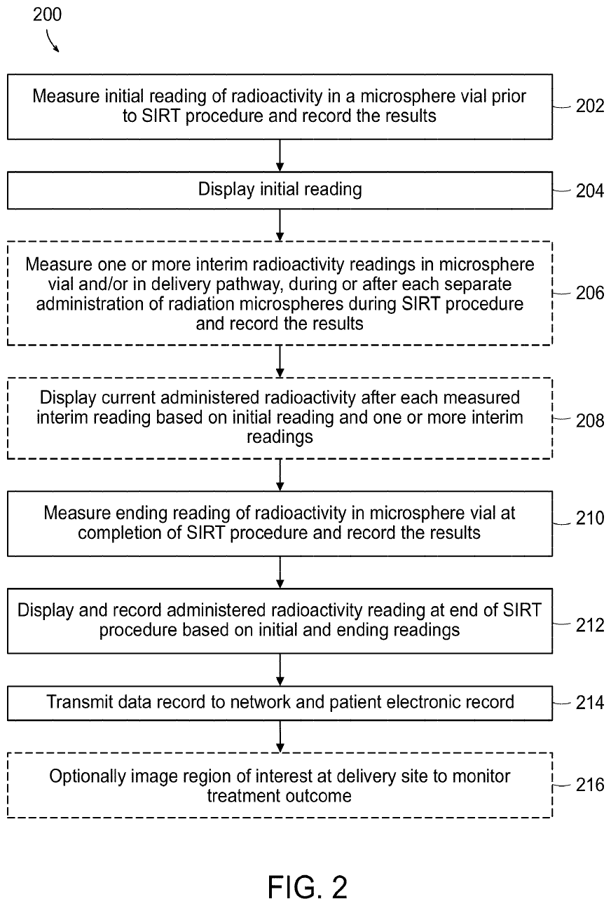

FIG. 2 is a flowchart of an exemplary sequence of steps used to provide real-time measuring of delivered radioactivity in an exemplary embodiment. The sequence begins with detector 106 measuring an initial reading of the radioactivity of the impregnated microspheres in vial 104 prior to beginning a SIRT or other medical procedure using radioactive isotopes and recording the results (step 202). The initial reading may then be displayed via scalar display 110 (step 204). One or more interim radioactivity measurements may then be taken by detector 106 after each separate administration of radioactive microspheres to patient 130 during a SIRT procedure with the results being stored in a non-volatile memory location (step 206). The interim readings may be used to display a current administered radioactivity value after each measured interim reading based on the initial reading of radioactivity and any subsequent interim readings. In this manner, the interventional radiologist may precisely control the amount of radioactivity being delivered to the patient so as to provide proper radioactive dosing for the SIRT or other procedure. An ending reading of radioactivity in the microsphere vial is taken at the completion of the SIRT or other medical procedure and the results stored (step 210). The total administered radioactivity is displayed based on the initial and ending readings (e.g.: for an initial reading x, ending reading y, administered radioactivity is equivalent to x-y) (step 212). The status of the microspheres and thus the distribution of radioactivity can optionally be monitored by imaging 214 of the region of interest.

A series of measurements were performed to record radioactivity in connection with certain-embodiments of the present invention. More particularly a study was conducted to observe the use of a portable Geiger Muller Counter (GMC) for real time monitoring of delivered radioactivity during TARE and compare its effectiveness to standard pre-delivery and post-delivery measurements. The procedure is described in detail below.

### Procedural Background

Typically, an AU monitors delivery by visual estimation of ⁹⁰Y microspheres in the vial, the degree of back pressure against the syringes, angiographic flow rates, and amount of remaining fluid (D5W) volume. Confirmation is drawn post-procedurally by a radiation survey by NM and Radiation Safety at which time corrections to deviations in therapy would require repeat catheterization. Therefore, to aid in real-time evaluation of received and delivered radioactivity, this procedure used a portable radiation detector such as a GMC for real time radioactivity monitoring during radioembolization and compared these measurements to the standard method using dose calibrators.

### Materials and Methods

This was a retrospective, observational, single center, consecutive case series evaluation of patients with primary and secondary liver cancer, who underwent TARE with 90Y resin microspheres (SIR-Spheres®, Sirtex Medical Limited, North Sydney, Australia). The decision to treat patients with TARE using ⁹⁰Y microspheres was approved after discussion in the multi-disciplinary liver tumor conference. Prescribed radioactivity was calculated by the authorized user and provided as a written directive to Nuclear Medicine. TARE was performed using the legacy and SIROS™ delivery apparatus with a portable Geiger Mueller counter (GMC) attached to them. In each case, the detector was placed inside the box or housing, and immediately adjacent to the location of the radioactivity vial.

### Radioactivity Measuring Devices

The GMC apparatus dose calibration was performed using the 451P Pressurized Radiation Detector (Fluke Biomedical Inc., Cleveland, OH, USA) and Capintec CRC-25R (Capintec Inc., Florham Park, New Jersey, USA). As noted previously, other radiation detectors such as scintillating detectors (liquid or polymer scintillator elements) or silicon photomultipliers can be used to measure delivered radioactivity as described herein. See for example, U.S. Pat. Nos. 8,614,420 and 11,054,530, the entire contents thereof being incorporated herein by reference. Solid state semiconductor detectors such as cadmium telluride sensors can also be used. Radiation detectors can be configured to discretely measure the radioactivity of each microsphere, for example, and can thereby accurately record the delivered dose. Geiger-Mueller detectors typically have a higher amplification to provide a higher signal to noise ratio and are thus effective at detecting lower energy pulses. Other detectors can also be used for calibration purposes. Note that ionization chambers, for example, have no inherent amplification and are thus more useful in other applications using a current mode of operation to detect different particles having varying levels of radioactivity.

### Calibration for NM Method

Reference standards were created by transferring the contents of a SIRTEX stock bottle into the delivery V-vial and decay correcting the contents from the reference date to the count date. The V-vial was assayed in the dose calibrator to confirm radioactivity. Initial calibration was performed by transferring 3 SIRTEX vials into v-vials that were each measured 10 times. The calibration conversion was chosen such that the average of the 30 measurements would yield an instruments response of 100* the known radioactivity (in GBq).

$$A ( 0.01 * GBq ) = ( Count rate ( c / s ) ) / ε ( c / ( 0.01 Gbq ) ) .$$

When the calibration value was determined, a fourth v-vial was pipetted to a predetermined radioactivity and assayed on the dose calibrator to verify the proper conversion of the instrument response to display 0.01*v-vial's radioactivity (GBq).

### Calibration of GMC Apparatus

The GMC was calibrated against a radioisotope dose calibrator. The dial settings were calibrated according to manufacturer standards. The count geometry of the ⁹⁰Y sphere vial was an important consideration when using the GMC. Microspheres were shaken prior to radioactivity measurement to prevent microsphere settling and ensure accurate radioactivity measurement. The plastic base of the legacy Sirtex™ delivery box was thinner than the range of ⁹⁰Y betas (1.1 cm) in Plexiglas, which may result in backscatter radiation depending on the administration table used.

### Pre-Procedural Radioactivity Calculations

Prescribed radioactivity was calculated principally using the single compartmental Medical Internal Radiation Dose (MIRD) model. After receiving the ⁹⁰Y SIR-spheres in a shipping vial from the manufacturer, radioactivity equivalent to the Written Directive (titrated to +10%) was drawn out by the technologist, measured using the dose calibrators, and labelled as ‘Drawn Radioactivity (NM)’. The Drawn Radioactivity (NM) was confirmed by recording measurements using a hand-held GMC around the vial in four different planes, all ninety degrees apart, as recommended by the manufacturer (this measurement is used to determine NM's estimated dose). An average of the four values was labelled as ‘Starting Radioactivity (NM)’. The portable in-procedure GMC was used to measure an alternate reading and was labelled as ‘Starting Radioactivity (GMC)’. All measurements with the GMC were performed with a well shaken vial of SIR-spheres because resin microspheres are known to settle to the bottom of the vial with the dextrose 5% water (D5W) forming a supernatant above.

### Intra-Procedural Radioactivity Measurements

The v-vial containing the drawn radioactivity and delivery box were transferred from the nuclear medicine department to the catheterization laboratory once target vessel catheterization had been achieved. The delivery housing was set up for administration. The v-vial was shaken to disperse the contents within, and a reading was recorded using the GMC and labelled as ‘Pre-treatment GMC reading’. Aliquots of SIR-spheres were administered via the delivery system tubing and pushed into the patient with a column of contrast OPTIRAY™ (loversol) Guerbet, Liebel-Flarsheim Company LLC, Raleigh, NC 27616). With every push of the injection, the reading on the GMC was recorded on a worksheet. For the delivery apparatus, when the downward trend of readings on GMC approached<10 centiBq where a Becquerel (Bq) is one decay per second for units of activity. SIR spheres were flushed using air to complete delivery of contents of the vial. The number of total injection pushes were recorded per patient. Following this final injection, radioactivity remaining in the vial as recorded by the GMC was labelled as ‘Final Radioactivity (GMC)’. At this point, SIR sphere administration was stopped assuming that most of the radioactivity had been administered, and target vessel flow characteristics were documented under fluoroscopy.

### Post-Procedural Radioactivity Measurements

Contents of the delivery box including the empty V-vial, contaminated syringe, tubing, pads, microcatheter and gloves were returned to the NM department in a Nalgene jar. A post-delivery radioactivity in the delivery box was measured by placing a hand-held GMC in the nuclear medicine hotlab around the vial in four different planes, all ninety degrees apart. The average of those values was labelled as ‘Final Radioactivity (NM)’. The radioactivity in the waste container was assayed by the in-procedure GM by placing the detector around the waste container in four different planes, all ninety degrees apart. The average of those values was labeled as ‘Waste Radioactivity (GMC)’.

### Variables

### Radioactivity Delivered

Radioactivity delivered was calculated by subtracting Final Radioactivity (NM) from Starting Radioactivity (NM). Radioactivity Delivered (GMC) was calculated by subtracting Final Radioactivity (GMC) and Waste Radioactivity (GMC) from Pre-treatment GMC reading.

$$Radio activity Delivered ( NM ) = Starting Radio activity ( NM ) - Final Radio activity ( NM ) \\ Radio activity Delivered ( GMC ) = Pre - treatment GMC reading ( GMC ) - Final Radio activity ( GMC ) - Waste Radio activity ( GMC ) .$$

### Waste Radioactivity

Final Radioactivity (NM) was the radioactivity measured at the end of the procedure by NM's hand-held GMC. Final Radioactivity (GMC) was the last value recorded in the IR suite at the end of the procedure less any generated waste as measured above. Final Radioactivity (NM) and Final Radioactivity (GMC) served as Waste radioactivity (NM) and Waste Radioactivity (GMC), respectively.

Comparison between Radioactivity Delivered (NM) and Radioactivity Delivered (GMC) Ratio of Radioactivity Delivered was calculated using Radioactivity Delivered (GMC) and Radioactivity Delivered (NM). A difference of +10% was considered acceptable keeping in view the manufacturers titration guidelines.

$$Percentage of Radio activity Delivered = ( Radio activity Delivered ( GMC ) ) / ( Radio activity Delivered ( NM ) ) \times 100.$$

GMC was used to treat 2 con-contiguous hepatic branches from the same vial. After starting the first round of SIR-sphere infusion, the downtrend observed on the GMC was used to estimate the real time radioactivity being delivered. After several rounds of administration, air was injected through the V-vial to administer the desired radioactivity. After attainment of a desired reading on the GMC, the procedure was stopped and the microcatheter was flushed two to three times before being disconnected from the delivery box and patient. Using radiation safety precautions, the microcatheter was removed coaxially into the catheter and both were removed en-bloc onto a sterile towel. A 3cc syringe was attached to the 5 French catheters to prevent leakage and a preliminary radiation safety check was performed via the representative of radiation safety. A new preloaded microcatheter system was connected to the A-tubing and advanced under fluoroscopic guidance into the desired hepatic artery location. At this point, a second round of TARE was carried out to infuse the desired radioactivity in a similar fashion with the down trending GMC readings serving as an estimate of the real time radioactivity being delivered. This system was used for patients with tumors that had a high likelihood of a dual supply as visualized and confirmed with hepatic cone beam CT imaging during the planning arteriogram. Post-TARE arteriograms were performed in the two treated locations to document the target vessel flow characteristics. After the procedure, non-contrast CT images were obtained for attenuation correction and for fusion with emission PET images. A series of overlapping emission PET images were obtained to show radioactivity distribution and localization in the treated locations as Mean tumor absorbed dose (D50). D50 was compared to MIRD Target Dose (Gy) Response to treatment was evaluated at 1-3 months using the modified RECIST (mRECIST) criteria for HCC as complete response (CR), partial response (PR), stable disease (SD) or progressive disease (PD).

### Subgroup Analysis

As part of a subgroup analysis, three groups of vials were made based on starting V-vial radioactivity: a) Vials with Low Radioactivity; (b) Vials with Intermediate Radioactivity, and (c) Vials with High Radioactivity. Groups were compared based on radioactivity delivered with each operator push, number of total pushes, as well as total amount of D5W used per procedure.

### Statistical Analysis

The database was de-identified and analysis was performed using SPSS, version 21.0 (Armonk, NY: IBM Corp) and Excel. Continuous variables were presented in the form of Mean±standard deviation and Range. Means were compared using 2-tailed, unpaired T-tests between 2 groups and ANOVA between 3 groups. As appropriate, all statistical tests and/or confidence intervals were performed at p=0.05.

### Results

### Pre-Procedurally

A total of ninety-six TARE administrations were performed in seventy-five patients. Legacy delivery box was used in 70% (68/96) of procedures and SIROS in 30% (28/96). Treated tumors included hepatocellular carcinoma (66/96, 69%), intrahepatic cholangiocarcinoma (12/96, 13%), and metastatic hepatic tumors (18/96, 19%). The prescribed radioactivity was 1.6 GBq±1.1 (0.2-6). The mean time between radioactivity preparation in NM and radioactivity delivered was 17 minutes±38.2 (0-210), resulting in mean radioactivity lost to decay of 0.02 GBq±0.16 (0.01-0.87). The median Starting Radioactivity recorded by NM and GMC was 1.7 GBq±1.2 (0.2-6.3) and 1.7±1.2 (0.2-6) (p-value=0.86).

### Intra-Procedurally

Infusions were performed by 3 board certified interventional radiologists with 8, 10 and 13 years of experience. Treatment characteristics are detailed in Table 1. Average number of pushes per procedure were 9±2 (2-17) with 6±3 (2-12) number of pushes being needed to deliver>90% of radioactivity. The radioactivity left in the vial measured by GMC before the final air shot was 0.28 GBq±0.41 (0.01-1.76) with an average radioactivity of 0.05 GBq±0.15 (0.01-1.07) being delivered with the air shot. Amount of fluid (D5W) injected through the pharmaceutical via the delivery system tubing was 28 ml±16 (5-80). 10% (10/96) and 4% (4/96) of all procedures were stopped early due to stasis and catheter blockage, respectively. More than 90% of radioactivity was delivered in 85% (82/96) of cases.

### Radioactivity Delivered

Radioactivity Dynamics are detailed in Table 2. There was no significant difference between Radioactivity Delivered as measured by NM and GMC (1.6 GBq±1.2 [0.2-5.7] vs 1.6 GBq±1.2 [0.2-5.8], p-value=0.93), respectively. There was no difference in radioactivity measured by NM and GMC among deliveries that were terminated due to stasis (10/96; (1.2 GBq±0.7 [0.5-2.7] vs 1.2 GBq±0.8 [0.4-2.7], respectively, p-value=0.99) nor among deliveries that were not terminated due to stasis (86/96; 1.6 GBq±1.2 [0.2-5.7] vs 1.6 GBq±1.2 [0.2-5.8], respectively, p-value=0.93).

### Post-Procedurally

Overall, there was no significant difference between waste radioactivity as measured by NM and GMC (0.2 GBq±0.2 [0-1.3] vs 0.1 GBq±0.2 [0-1.2], p-value=0.33). There was also no significant difference between waste radioactivity as measured by NM and GMC between cases with stasis (0.3 GBq±0.3 [0-0.8] vs 0.4 GBq±0.2 [0-0.8], p-value=0.96), nor among cases without stasis (0.1 GBq±0.2 [0-1.3] vs 0.1 GBq±0.2 [0-1.2], p-value=0.24).

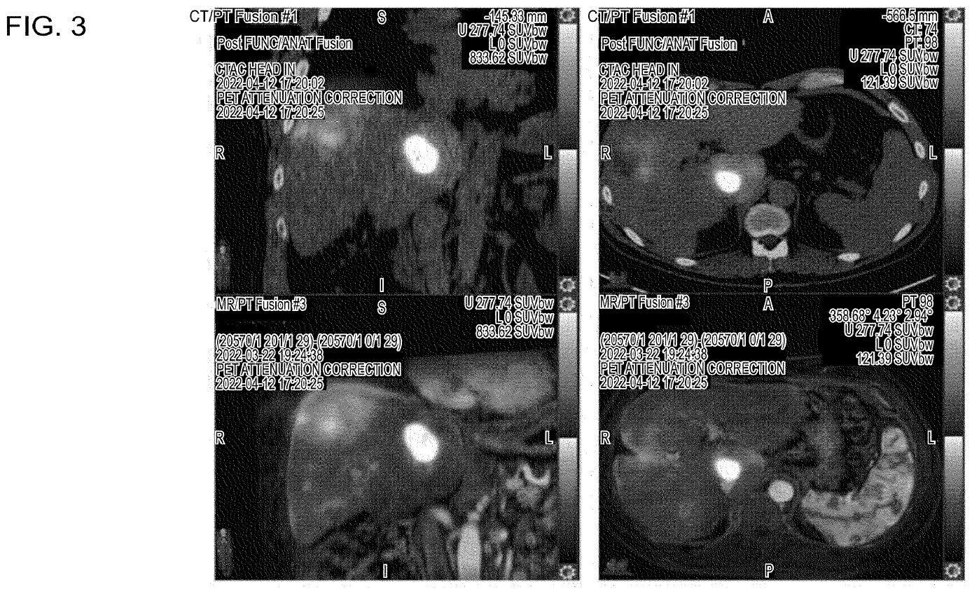

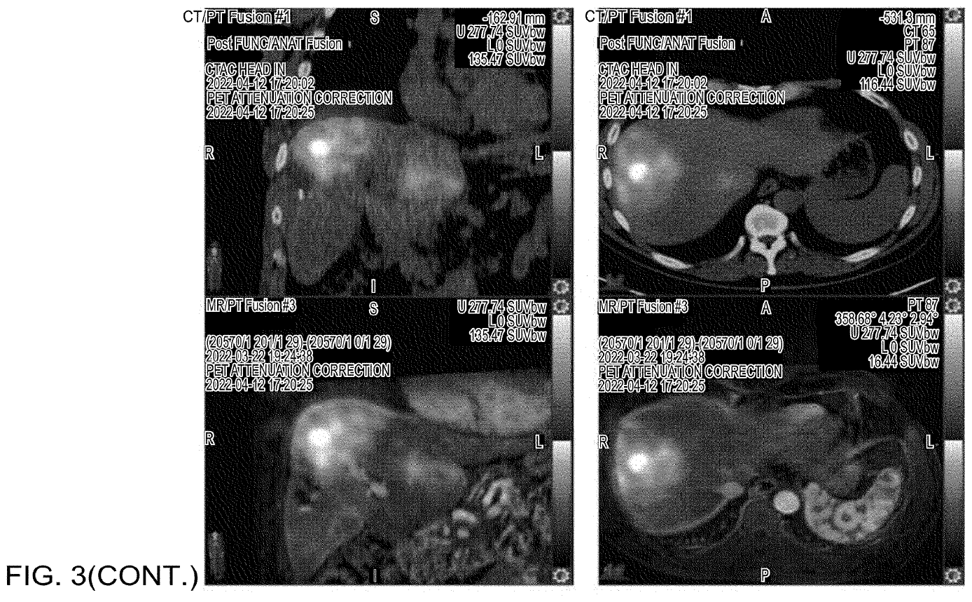

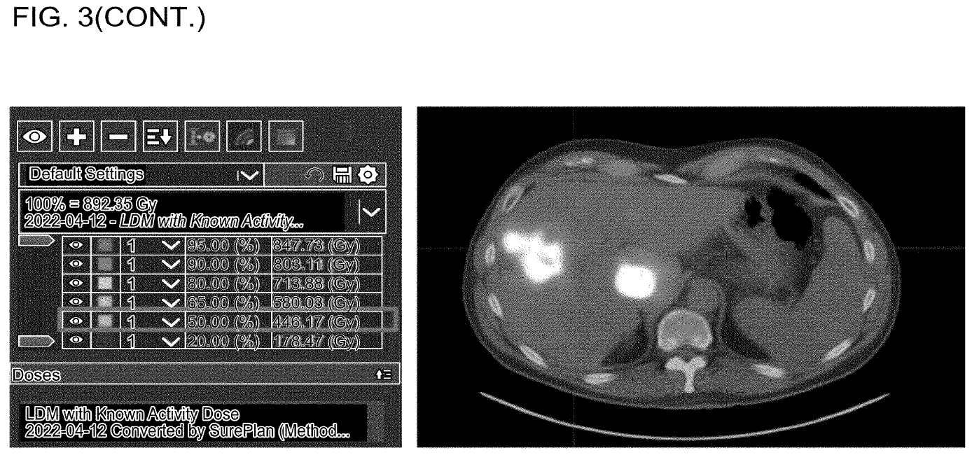

For this measurement, resin microspheres from the same vial were injected into two injection sites during the same procedure in 10 patients with 14 tumors (single tumors: 6/10 patients [60%]; dual tumors: 4/10 [40%] patients) (see Table 3A and 3B, FIG. 3). Average WD was 1.7 GBq±0.7 (1-3.2) and average radioactivity per infusion was 0.9 GBq±0.5 (0.2-1.8). There was no difference between prescribed the tumor absorbed dose on prescription dosimetry and D50 on PET computed tomography (CT) (235 Gy #53 (200-350) vs 264 Gy #168 (99-636), respectively; p=0.29). Post-treatment imaging at 1-3 months (average 96 days) was available for 8 patients (11 tumors) and demonstrated complete response in 72% (8/11), partial response in 18% (2/11) and stable disease in 9% (1/11).

### Subgroup Analysis

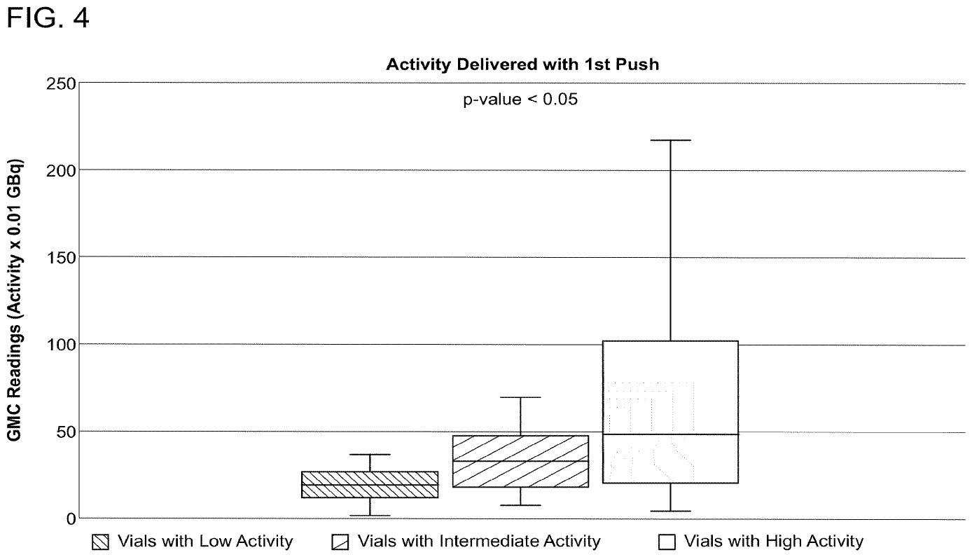

Among the vials used in 96 procedures, 24% were labelled in the low radioactivity group (Group 1) (0.66 GBq±0.2 [0.23-0.98]), 49% in intermediate radioactivity group (Group 2) (1.36 GBq±0.29 [1-1.99]), and 27% in high radioactivity group (Group 3) (3.33 GBq #1.34 [2-5.95]). On average, the amount of radioactivity delivered with each push was significantly different among the three groups (G1 vs. G2 vs. G3) (0.09 GBq vs. 0.15 GBq vs. 0.36 GBq; P<0.001). The amount of radioactivity delivered with the first push was significantly higher in vials with higher radioactivity compared to lower radioactivity (0.71 GBq [G3] vs. 0.33 GBq [G2] vs. 0.18 GBq [G1]; P<0.001) (FIG. 4). However, the proportion of radioactivity delivered with each push was comparable between all groups (13% [G1] vs. 11% [G2] vs. 11% [G3]; P: 0.3).

This procedure tracked the use of real-time radioactivity monitoring during delivery of resin microspheres in TARE. The real-time delivery measurement using a portable GMC showed no difference in starting, delivered or waste radioactivity measurement compared to a standard NM measurement method. This portable device after appropriate calibration obtains real-time dynamic measurements. Embodiments provide awareness of delivered radioactivity in real time that will better inform interventional radiologists of completion of radioactivity delivery compared to the current use of visual estimation of microsphere density. Thus, operators can confirm before terminating the procedure whether the entire radioactivity dose has been delivered from the vial.

In this procedure, there was an average radioactivity decay of 0.02 GBq±0.16 GBq with some vials experiencing decay as high as 0.87 Gbq between the nuclear medicine dose draw and initiation of radioactivity delivery. Putting this into perspective, a smaller radioactivity vial can experience much higher radioactivity loss and residual radioactivity compared to a larger radioactivity vial, with supportive data existing in past in ex-vivo studies.

Another challenge when managing ⁹⁰Y microspheres between NM and the IR suite, is the inability to identify the vials prior to infusion. This lack of a secondary check in the IR suites may increase risk of vial mixing and wrong patient delivery. As noted previously, the ME report by the NRC (2008-2017) showed that 32% of medical events occurred due to mixing up of written directives which led to patients receiving incorrect prescribed doses. This risk may increase in hospitals which experience a higher volume of TARE procedures where multiple patients may be treated on the same day. A real-time radioactivity monitor using a GMC in the catheterization laboratory can help overcome this challenge by identifying and confirming whether the prescribed radioactivity in the vial matches the WD for the patient being treated. Mean residual activities of 4.0% (median 3.6%, range 1.2%-6.6%) and 3.4% (median 3.4%, range 0.9%-8.8%) have been reported for resin and glass microspheres, respectively. Radioactivity output from the GMC offered real time feedback of radioactivity delivered with each push, thereby allowing operators to regulate the delivery of the radiation microspheres. In the treatment algorithm, operators stopped pushing into the syringe's plunger when radioactivity ceased to downtrend on the GMC interface, as opposed to the conventional step of waiting to feel a backpressure against the plunger or a visual estimation of remaining microspheres. As mentioned in the NRC reports, such conventional steps have historically led to higher residual activities post-treatment. Embodiments of the present invention may aid in decreasing residual radioactivity, as it allowed operators to re-adjust the micro catheter positioning which often relieved the backpressure and allowed further delivery of ⁹⁰Y spheres, evidenced by the resumption of radioactivity downtrend on the GMC interface.

Once accuracy of method was determined, the approach was applied by using a single vial to treat 2 non-contiguous hepatic branches from the same vial. Using patient five as an example: 0.4 GBq was delivered into segment 7, after which the first microcatheter was removed using radiation safety precautions and a second microcatheter was inserted into segment 1 (caudate lobe) and an additional 1.4 GBq was delivered. Real time radioactivity monitoring enabled accurate splitting of the single vial between the two treatment locations. Fused PET/CT images were obtained about an hour after administration that demonstrated 2 areas of radioactivity (see FIG. 3). This novel procedure may offer operators the opportunity to treat 2 non-contiguous hepatic segments using a single vial and can be confirmed on ⁹⁰Y bremsstrahlung SPECT/CT post treatment.

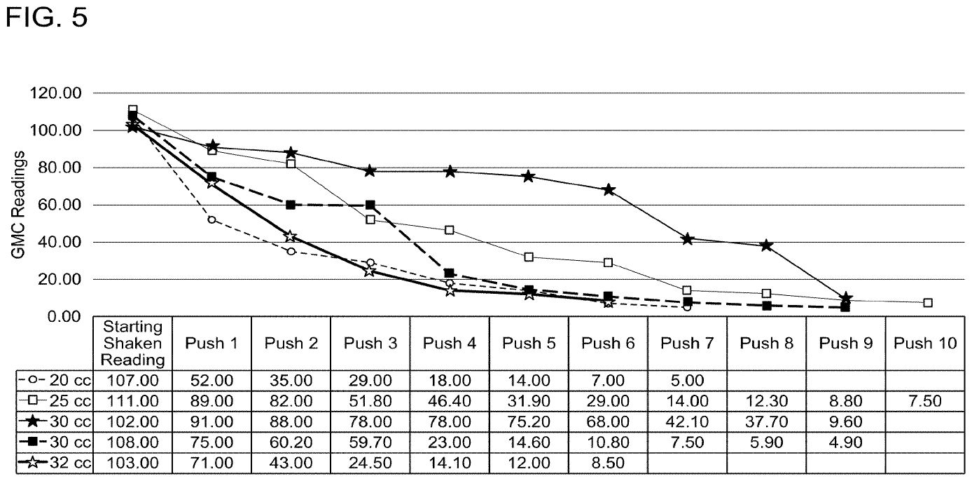

While the GM results match those of NM, there are additional factors to consider. Physical properties of the medium in the v-vial may influence settling rates of the resin microspheres which can lead to the path to the detector getting longer or shorter, with the former causing more attenuation than the latter. In this analysis, microspheres were found to settle quicker (longer path) with saline than D5W (shorter path). Furthermore, vials with higher radioactivity deliver more radioactivity with each push compared to vials with lower activities. In this analysis, vials with higher starting activities delivered four times more radioactivity in the first push than vials with lower starting activities (0.71 GBq vs 0.18 GBq) (Table 4). Moreover, the effects of operator's preference and amount of pressure applied while injecting on Delivered Radioactivity is also largely uncertain. When comparing the number of total pushes and amount of fluid (D5W) used among 4 similar vials of 1GBq, some vials required as low as 6 pushes, while others as high as 10 to deliver>90% of radioactivity with overall D5W being 20-32 cc (see FIG. 5). Thus, external factors like amount of pressure applied may vary among operators and can ultimately influence the amount of radioactivity being delivered per push. Variability in applied pressure and amount of radioactivity delivered with each push recommends caution in circumstances when a larger radioactivity vial may be used for delivering a smaller desired radioactivity.

A portable real time radioactivity monitoring GMC device demonstrates improved radioactivity measurements compared to the standard practice of using ion chambers for verification prior to delivery of radioactivity using resin microspheres. Routine use of this technique can save time and radioactivity lost to decay, decrease risk of radioactivity spillage and line losses, and allows operators to treat multiple segments or lobes with the same vial.

Supporting Tables: Table 1: Treatment Characteristics

| Variables | Mean ± SD (Range) |
|---|---|
| Number of Patients | 75 |
| Number of TARE | 96 |
| Mean Age (years)* | 68.7 ± 11.6 (16-96) |
| Female Subjects* | 28% (21/75) |
| Primary Hepatic Tumor (n = 78/96, 81%) | |
| HCC | 69% (66/96) |
| IHCC | 13% (12/96) |
| Secondary Tumor (n = 18/96, 19%) | |
| CRC | 9% (9/96) |
| Other | 9% (9/96) |
| Written Directive (GBq) | 1.6 ± 1.1 (0.2-6) |
| Number of Pushes^{†} | 9 ± 2 (2-17) |
| Number of Pushes to deliver 90% of the | 6 ± 3 (3-12) |
| radioactivity | |
| Amount left in vial before air shot^{††} (GBq) | 0.28 ± 0.41 (0.01-1.76) |
| Amount delivered with air shot (GBq) | 0.05 ± 0.15 (0.01-1.07) |
| Amount of D5W | 28 ± 16 (5-80) |
| Stasis | 10% (10/96) |

*Per patient analysis
TARE: Transarterial Radioembolization; HCC: Hepatocellular Carcinoma; IHCC: Intrahepatic Cholangiocarcinoma; Mets: Metastasis; CRC: Colorectal Carcinoma; D5W: Dextrose 5% in water; GBq: gigabecquerel
^{†}Pushes refer to aliquots of microspheres injected
^{††}Air shot refers to the use of air to push the last aliquot into the microcatheter

TABLE 2
Radioactivity Dynamics (per-procedure analysis)
Overall (n = 96)

| Variables | NM* | GM* | P-value |
|---|---|---|---|
| Starting radioactivity (GBq) | 1.7 ± 1.2 (0.2-6.3) | 1.7 ± 1.2 (0.2-6) | 0.86 |
| Waste Radioactivity (GBq) | 0.2 ± 0.2 (0-1.3) | 0.1 ± 0.2 (0-1.2) | 0.33 |
| Delivered radioactivity (GBq) | 1.6 ± 1.2 (0.2-5.7) | 1.6 ± 1.2 (0.2-5.8) | 0.93 |

Non-Stasis (n = 86/96, 90%)

| Variables | NM | GM | P-value |
|---|---|---|---|
| Starting radioactivity (GBq) | 1.7 ± 1.3 (0.2-6.3) | 1.7 ± 1.3 (0.2-6) | 0.93 |
| Waste Radioactivity (GBq) | 0.1 ± 0.2 (0-1.3) | 0.1 ± 0.2 (0-1.2) | 0.24 |
| Delivered radioactivity (GBq) | 1.6 ± 1.2 (0.2-5.7) | 1.6 ± 1.2 (0.2-5.8) | 0.93 |

Stasis (n = 10/96, 10%)

| Variables | NM | GM | P-value |
|---|---|---|---|
| Starting radioactivity (GBq) | 1.5 ± 0.7 (0.5-2.8) | 1.6 ± 0.8 (0.5-2.8) | 0.66 |
| Waste Radioactivity (GBq) | 0.3 ± 0.3 (0-0.8) | 0.4 ± 0.2 (0-0.8) | 0.96 |
| Delivered radioactivity (GBq) | 1.2 ± 0.7 (0.5-2.7) | 1.2 ± 0.8 (0.4-2.7) | 0.99 |

*Mean ± SD (Range)
NM: Nuclear Medicine; GM: Geiger Muller; GBq: gigabecquerel; Gy: Gray

TABLE 3A
Analysis (Patient and Procedure Characteristics)

| Characteristics | Distribution |
|---|---|
| n | 10* |
| Age | 68 ± 10 (47-80) |
| Female | 30% (3/10) |

Cancer

| | |
|---|---|
| HCC | 60% (6/10) |
| IHCC | 20% (2/10) |
| mCRC | 20% (2/10) |

Delivery Box

| | |
|---|---|
| Legacy | 60% (6/10) |
| Siros | 40% (4/10) |
| Prescribed TAD (Gy) | 235 ± 53 (200-350) |

Radioactivity Dynamics

| | |
|---|---|
| Written Directive (GBq) | 1.7 ± 0.7 (1-3.2) |
| Overall Radioactivity Delivered NM (GBq) | 1.77 (0.98-2.06) |
| Overall Radioactivity Delivered GM (GBq) | 1.85 (1.08-2.02) |
| Mean Radioactivity Delivered In Single | 0.9 ± 0.5 (0.2-1.8) |
| Infusion (GBq) | |

PET-CT Analysis

| | |
|---|---|
| Mean D₅₀ achieved (Gy) | 264 ± 168 (99-636) |

*10 patients with 14 tumors
HCC: Hepatocellular Carcinoma; IHCC: Intrahepatic Cholangiocarcinoma; Mets: Metastasis; TAD: Tumor Absorbed Dose (Gy); NM: Nuclear Medicine; GM: Geiger Muller; GBq: gigabecquerel; Gy: Gray; D₅₀: minimum absorbed dose delivered to 50% of the tumor volume

TABLE 3B
Analysis (Per-patient analysis)

| 200 Gy | 2.2 | Gbq | 2.5 GBq | S6 | 1.2 GBq | 512 Gy | S5/8 | 1.1 | GBq | 175 Gy | CR | CR |
|---|---|---|---|---|---|---|---|---|---|---|---|---|
| 200 Gy | 1.8 | Gbq | 1.8 GBq | S7 | 1.4 GBq | 139 Gy | S1 | 0.4 | GBq | 446 Gy | CR | CR |
| | | | | | | | Caudate | | | | | |
| 200 Gy | 2 | Gbq | 2.03 GBq | S5/6 | 1.3 GBq | 150 Gy | Branch off | 0.73 | GBq | 231 Gy | CR | CR |

S7^{†}

TABLE 4
Subgroup Analysis - Radioactivity Delivered with each Push based on Starting Vial Radioactivity

| | Small Vials | Vials | Intermediate | Vials | Large Vials | Vials | |
|---|---|---|---|---|---|---|---|
| | (0.6 ± 0.2 | Terminated | Vials (1.3 ± | Terminated | (3 ± 1.4 | Terminated | |
| Activity (GBq) | (0.2-1)) | (n = 24) | 0.3 (0.5-1.8)) | (n = 46) | (1.2-6.5)) | (n = 26) | P-value |
| Shaken Reading | 0.66 GBq ± 0.2 | 0% | 1.36 GBq ± 0.29 | 0% | 3.33 GBq ± 1.34 | 0% | <0.001 |
| Activity Pushed 1 | 0.18 GBq ± 0.1 | 0% | 0.33 GBq ± 0.18 | 0% | 0.71 GBq ± 0.59 | 0% | <0.001 |
| Activity Pushed 2 | 0.1 GBq ± 0.08 | 0% | 0.22 GBq ± 0.19 | 0% | 0.42 GBq ± 0.28 | 0% | <0.001 |
| Activity Pushed 3 | 0.09 GBq ± 0.06 | 0% | 0.18 GBq ± 0.13 | 0% | 0.49 GBq ± 0.3 | 4% | <0.001 |
| Activity Pushed 4 | 0.06 GBq ± 0.07 | 8% | 0.17 GBq ± 0.18 | 0% | 0.45 GBq ± 0.27 | 4% | <0.001 |
| Activity Pushed 5 | 0.11 GBq ± 0.11 | 13% | 0.11 GBq ± 0.08 | 7% | 0.31 GBq ± 0.19 | 12% | <0.001 |
| Activity Pushed 6 | 0.04 GBq ± 0.03 | 25% | 0.08 GBq ± 0.07 | 11% | 0.29 GBq ± 0.24 | 12% | <0.001 |
| Activity Pushed 7 | 0.04 GBq ± 0.05 | 42% | 0.07 GBq ± 0.07 | 20% | 0.18 GBq ± 0.2 | 23% | 0.002 |
| Activity Pushed 8 | 0.02 GBq ± 0.02 | 54% | 0.05 GBq ± 0.05 | 33% | 0.22 GBq ± 0.26 | 35% | <0.001 |
| Activity Pushed 9 | 0.02 GBq ± 0.02 | 75% | 0.06 GBq ± 0.06 | 50% | 0.17 GBq ± 0.32 | 42% | 0.15 |
| Activity Pushed 10 | 0.04 GBq ± 0.02 | 83% | 0.02 GBq ± 0.02 | 76% | 0.08 GBq ± 0.09 | 69% | error** |
| Activity Pushed 11 | n/a | 100% | 0.07 GBq ± 0.06 | 91% | 0.06 GBq ± 0.05 | 92% | error |
| Activity Pushed 12 | n/a | 100% | 0.03 GBq ± 0.02 | 93% | 0.09 GBq ± 0.08 | 92% | error |
| Activity Pushed 13 | n/a | 100% | 0.05 GBq ± 0.04 | 96% | 0.02 GBq ± 0.01 | 92% | error |
| Activity Pushed 14 | n/a | 100% | 0.05 GBq ± 0 | 98% | 0.01 GBq ± 0 | 92% | error |
| Activity Pushed 15 | n/a | 100% | 0.04 GBq ± 0 | 98% | 0.01 GBq ± 0 | 96% | error |
| Activity Pushed 16 | n/a | 100% | n/a | 100% | n/a | 100% | error |

*Mean ± (SD)
**insufficient sample size
^{†}8-10 operator pushes for majority of the vials (60%, 57/96)
^{††}Not shown (Per-vial group): Total pushes (p = 0.4), Pushes needed to deliver >90% of vial radioactivity(p = 0.3), Volume of D5 (p = 0.1)

Portions or all of the embodiments of the present invention may be provided as one or more computer-readable programs or code embodied on or in one or more non-transitory mediums. The mediums may be, but are not limited to a hard disk, a compact disc, a digital versatile disc, ROM, PROM, EPROM, EEPROM, Flash memory, a RAM, or a magnetic tape. In general, the computer-readable programs or code may be implemented in any computing language.

Since certain changes may be made without departing from the scope of the present invention, it is intended that all matter contained in the above description or shown in the accompanying drawings be interpreted as illustrative and not in a literal sense. Practitioners of the art will realize that the sequence of steps and architectures depicted in the figures may be altered without departing from the scope of the present invention and that the illustrations contained herein are singular examples of a multitude of possible depictions of the present invention. The foregoing description of example embodiments of the invention provides illustration and description, but is not intended to be exhaustive or to limit the invention to the precise form disclosed. Modifications and variations are possible in light of the above teachings or may be acquired from practice of the invention. For example, while a series of acts has been described, the order of the acts may be modified in other implementations consistent with the principles of the invention. Further, non-dependent acts may be performed in parallel.

1. A microsphere delivery apparatus for real-time measuring of radioactivity delivery to a patient, comprising:
a delivery housing containing:
a delivery apparatus configured to deliver radiation emitting particles along a delivery path from a container to a patient during radiation therapy; and
a detector that detects radiation emitted by the particles to perform real-time monitoring of administered radioactivity delivered to the patient, the detector including:
a radiation probe sized for positioning in a detector holder located at a distance from the container, and
a memory connected to the radiation probe and configured to store a probe output in units of radioactivity.

2. The apparatus of claim 1 wherein the radiation probe is a Geiger Mueller counter that is removably insertable into the detector holder at a fixed distance from the container.

3. The apparatus of claim 1 wherein the radiation probe comprises a solid state detector.

4. The apparatus of claim 1 wherein the radiation emitting particles comprise microspheres that can optionally be in a size range of 15-60 microns.

5. The apparatus of claim 1 further comprising a controller that controls data transmitted to a display and a pump that controls a flow rate of the particles from the container.

6. The apparatus of claim 1 wherein the delivery housing further comprises a lead element.

7. The apparatus of claim 4 wherein the microspheres comprise a radioisotope yttrium-90 (⁹⁰Y) or Holmium-166 (¹⁶⁶Ho).

8. The apparatus of claim 1 wherein the detector is configured to be positioned within an opening of 50 square cm or less for the detector holder within the delivery housing.

9. The apparatus of claim 1 wherein the detector is configured to fit within a volume of less than 300 cubic cm within the delivery housing.

10. The apparatus of claim 4 wherein the radiation emitting microspheres are resin microspheres or glass microspheres.

11. (canceled)

12. The apparatus of claim 4 wherein the radiation emitting microspheres are biodegradable.

13. The apparatus of claim 1 wherein the detector is configured to measure an initial reading of radioactivity in the container prior to an initiation of delivery and to measure an administered radioactivity reading at the end of a selective internal radiation therapy (SIRT) procedure, the detector being connected to an electronic display configured to display radioactivity readings.

14. (canceled)

15. (canceled)

16. The apparatus of claim 1 further comprising a syringe or a pump to actuate fluid flow through the delivery apparatus and into a delivery catheter for delivery into a patient and wherein administration housing is an least partially transparent for viewing of the delivery apparatus comprising one or more tubes for transport of fluid including the radiation emitting particles.

17. (canceled)

18. The apparatus of claim 16 wherein the syringe or pump is coupled to a first channel to deliver a fluid into the container.

19. The apparatus of claim 18 further comprising a second channel wherein fluid exits the container with radioactive particles in the fluid wherein the second channel is fluidly coupled to a catheter configured to be inserted into the patient and optionally further comprising a second syringe or pump that is coupled to a third channel to actuate delivery of a contrast agent to the patient.

20. (canceled)

21. (canceled)

22. (canceled)

23. The apparatus of claim 18 wherein the first channel comprises a first disposable tube.

24. The apparatus of claim 19 wherein the second channel comprises a second disposable tube and wherein the third channel comprises a third disposable tube.

25. (canceled)

26. The apparatus of claim 1 further comprising a controller that generates a data record for a selected time interval during a delivery procedure, the data record including patient data, a time stamp and radioactivity data measured during the time interval.

27. (canceled)

28. The apparatus of claim 1 further comprising a processor, the processor being programmed with a software module to operate a delivery procedure and further comprising a network interface for transmitting radioactivity data to a network for recording with a patient electronic medical record.

29. The apparatus of claim 28 wherein the software module records a prescribed radiation dose for a first delivery procedure for a patient from a first vial of radiation emitting particles at a first location.

30. The apparatus of claim 29 wherein the software module records a further prescribed dose for a second delivery procedure for the patient from the first vial at a second location.

31. The apparatus of claim 1 further comprising a pressure sensor to measure a fluid pressure of a delivery fluid used to deliver radiation emitting particles to the patient.

32. The apparatus of claim 1 further comprising a flow sensor to measure a flow rate of a delivery fluid that delivers radiation emitting particles to the patient.

33. (canceled)

34. The apparatus of claim 5 wherein the display is configured to display patient identification for the procedure and a prescribed radiation dose for the procedure.

35. The apparatus of claim 1 wherein the memory records a first level of radioactivity in the container prior to the procedure and a second level of radioactivity in the container at the end of the procedure and wherein a processor computes a level of decay of radioactivity in the container as a function of time, the processor receiving monitoring data to record a level of delivered radioactivity at a location within the patient and exports a data record of a delivery procedure to an electronic medical record of the patient.

36. (canceled)

37. (canceled)

38. (canceled)

39. The apparatus of claim 28 wherein the device communicates to an external portable communication device a status of the delivery procedure.

40. A method for real-time measuring of radioactivity during radiation microsphere transport, comprising:
measuring an initial reading of radioactivity in a microsphere source prior to beginning radiation emitting microsphere transport during with a radiation detector enclosed within a housing, the radiation detector being connected to an electronic display that displays an output in units of radioactivity, the housing including a fluid flow transfer path coupled to the microsphere source;
recording the initial reading; and
measuring an ending reading of radioactivity in the microsphere source after transport of radiation emitting microspheres from the housing along the fluid flow path.

41. The method of claim 40 further comprising displaying an transferred radioactivity reading at the end of a fluid transfer period based on the initial reading and the ending reading.

42. The method of claim 40, further comprising:
measuring an interim reading of radioactivity in the microsphere vial after each separate transfer of radiation micropsheres from the housing; and
displaying a current radioactivity reading following each measured interim reading, the current administered radioactivity reading based on the initial reading and one or more interim readings.

43. The method of claim 42, further comprising:
performing the measuring of the initial, interim and ending readings of radioactivity during a transfer procedure that is performed using the same microsphere vial to transfer radiation microspheres to two or more separate transfer periods.

44. The method of claim 40 wherein the radiation emitting microspheres are resin microspheres or glass microspheres.

45. (canceled)

46. The method of claim 40 wherein the radiation emitting microspheres are biodegradable.

47. The method of claim 40 wherein the radiation detector comprises a radiation probe of a Geiger Mueller counter.

48. The method of claim 40 wherein the radiation emitting microspheres include radioisotope yttrium-90 (⁹⁰Y) or Holmium-166 (¹⁶⁶Ho).

49. The method of claim 40 further comprising controlling an operation of the radiation detector with a programmable controller connected to a radiation detector and a memory and communicating a data record of transferred radioactivity to a network interface to record a transfer in an electronic record.

50. (canceled)

51. The method of claim 40 wherein the housing has a least a region of transparent material to view a fluid flow path through the housing and further comprising transferring radiation emitting microspheres having a size in a range of 15-60 microns.

52. (canceled)

53. (canceled)

54. The method of claim 40 further comprising actuating fluid flow with a pump or syringe to control fluid flow through the housing and detecting radiation with a further radiation detector to detect radiation at a separate location in or proximate to the housing and displaying a level of radioactivity on a display coupled to the housing to monitor radioactivity in a container in which the microspheres are located, the container receiving a fluid from a first flow channel and delivering fluid in a second flow channel.

55. (canceled)

56. The method of claim 40 further comprising storing monitored radioactivity levels in a memory mounted to the housing and transferring stored radioactivity data to at external device with a communication network.

57. (canceled)

58. The method of claim 40 further comprising detecting radiation with a solid state radiation detector.

59. The method of claim 40 further comprising programming a controller with a software module to actuate a fluid flow to transfer radiation microspheres through the housing.

60. The method of claim 40 further comprising sensing a fluid pressure and/or a flow rate of a fluid through the housing.
